(19) United States
(12) Patent Application Publication
(10) Pub. No.: US 2004/0019308 A1
(43) Pub. Date: Jan. 29, 2004

(54) Strap for treating a deformed toe or finger

(76) Inventor: James C.Y. Chow, Mount Vernon, IL (US)

Correspondence Address: POLSTER, LIEDER, WOODRUFF & LUCCHESI, 763 SOUTH NEW BALLAS ROAD, ST. LOUIS, MO, 63141-8750, US

(21) Appl. No.: 10/206,467

(22) Filed: Jul. 25, 2002

(51) Int. Cl. A61F 5/00; A61F 13/00; A61F 13/06

(52) U.S. Cl. 602/61; 602/22; 602/30

(57) ABSTRACT

A support (10) worn on a person's foot (F) or hand to correct a deformity to a toe (T2) or finger. A first sleeve (12) of a support material is sized to fit over the adjacent toe (T1). A second sleeve (14) of the material is sized to fit over another toe (T3). A band (16) of material extends between the two sleeves. The band of material fits over the deformed toe when the support is worn and holds the deformed toe in a position in which it does not overlay the adjacent toe but extends generally side-by-side with it. Another embodiment of the support can be worn on a person's hand to treat a deformed finger. The support can be incorporated into a sock (100) or glove (110).

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

This invention relates to the treatment of a deformed toe or finger, and more particularly, to a strap or brace which is conveniently worn by the person with the deformity to correct the deformity.

A toe deformity occurs when one of the toes on a person's foot, usually one of the middle toes, rather than extending generally outwardly from the end of the foot, extends at an angle. This condition can be congenital, caused by degenerative changes to the foot over time, or result from an acute injury to the foot such as a fractured toe. When it occurs, the deformed toe presses against the side of an adjacent toe, moving up over, or down under, that toe. Sometimes when the toe is injured, the outer end of the toe will bend from the PIP joint, perpendicular to the inner portion of toe, creating a “hammer” toe condition. With the big toe, if a bunion occurs, the toe may turn out, away from the adjacent toe. This is referred to as a hallux valgus condition.

When a deformity happens, besides the pain caused by the pressure of the toe being out of its normal position, the skin around the toe is stretched and often breaks resulting in sores and creating a risk of infection. Sometimes, this can lead to amputation. Besides being extremely painful, the sufferer is often prevented from wearing normal shoes and socks, and must instead wear special footwear which can be expensive. The condition also significantly limits the person's range of activities.

A similar condition can occur with a finger, particularly as the result of an injury such as a fracture. Again, the person's ability to perform many activities is curtailed because of their ability to use their hand to grasp and hold objects, without injuring the deformed finger, is significantly lessened.

One treatment for these conditions is corrective surgery. Short of that, a more conservative approach is to restrain the toe or finger in a relatively normal position and hold it in that position for a significant period of time. Restraining a toe or finger, postoperatively, can also aid in recovery from a surgery.

Up until now, there has not been an appliance available to assist a physician in this treatment regimen. Usually, the physician has had to tape toes or fingers together with the deformed toe or finger being tightly held in a desired position. Tape is messy. It sticks to the skin, irritates the skin, and can pull off skin when removed. Again, this invites the risk of sores and infections. Further, tape tends to wrinkle as it is wound between the toes or fingers. Wrinkles rub against the skin and cause sores. A soft cloth material could be used, but this requires the use of clamps, a Velcro® material, or the like to provide sufficient force to hold the toe or finger in place. These also add bulk to the cloth, making it less comfortable to wear and restricting the activities of the patient. For a person with a deformed toe, the inability to conveniently and comfortably restrain the deformed toe also limits the types of shoes or socks the patient can wear.

### BRIEF SUMMARY OF THE INVENTION

In accordance with the invention, briefly stated, a toe or finger brace or support comprises a band of a smooth, elastic material which fits over or about the deformed toe or finger. On opposite ends of the band loops of material are formed into sleeves. The sleeves fit onto toes or fingers on each side of the deformed toe or finger. The band of material suppresses the toe or finger so when the support is worn, the deformed toe or finger is held in place in a normal side-by-side toe position rather than overlying or underlying the adjacent toe or finger. The support is left in place as long as desired, does not unduly restrict activities of the patient, and for persons suffering from a deformed toe, allows the patient to wear normal shoes and socks.

The support is low cost, re-usable, and is available in different sizes and shapes.

Other objects and features will be in part apparent and in part pointed out hereinafter.

### BRIEF DESCRIPTION OF THE SEVERAL VIEWS OF THE DRAWINGS

In the drawings, FIG. 1A a foot with a deformed (overlying) toe, and FIG. 1B a deformed (underlying) toe;

FIG. 2 is a top view of a first embodiment of a toe support of the present invention;

FIGS. 3A-3C are elevation views of three constructions of the first embodiment;

FIGS. 4A-4C illustrate use of the toe support to retain a deformed toe in a normal position; and,

FIG. 5 is a top view of a second embodiment of the toe support;

FIG. 6 is an elevation view thereof;

FIG. 7 illustrates how the second embodiment of the support is worn;

FIG. 8 is an elevation view of a third embodiment of the support;

FIG. 9 is a top view of a fourth embodiment of the support;

FIG. 10 illustrates an embodiment of the invention in which the insides of the sleeves are padded;

FIGS. 11 and 12 are respective plan and elevation views of another embodiment of the invention employing sleeves fitting over the deformed toe or finger, and adjacent toes or fingers;

FIG. 13 illustrates use of the support of FIGS. 11 and 12 to retain a deformed toe or finger in a normal position;

FIGS. 14 and 15 are respective plan and elevation views illustrating another embodiment of the invention in which the sleeves are immediately attached to each other rather than by a band;

FIGS. 16 and 17 are respective plan and elevation views of a variation of the embodiment of FIGS. 14 and 15;

FIGS. 18 and 19 are respective plan and elevation views of a sock in which the toe strap of the invention is incorporated; and,

FIGS. 20 and 21 are similar views for a glove worn by a person suffering from a deformed finger.

Corresponding reference characters indicate corresponding parts throughout the drawings.

### DETAILED DESCRIPTION OF THE INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

Referring to the drawings, the present invention is directed to the correction of a deformed digit, whether the digit be a pedal digit (i.e., a person's toe), or a finger of their hand. FIGS. 1A and 1B illustrate deformities to the toes of a foot F in a middle toe T2-T4 of the foot, rather than extending in a side-by-side, generally parallel relationship with the other toes of the foot, overlies (FIG. 1A) or underlies (FIG. 1B) an adjacent toe of the foot. For conditions effecting big toe T1 or little toe T5, surgery is usually the only option to correct the condition. For the intermediate toes T2-T4, the present invention is for a non-operational (or in some circumstances post-operative) treatment to correct the problem. While not shown in the drawings, similar deformities occur with the fingers. It will be understood that that while the deformity shown is a common deformity, other conditions to the toes and fingers are also correctable using the supports of the present invention. In the various embodiments described hereinbelow, it will be understood by those skilled in the art, that various of the toe strap configurations shown are readily adapted for wearing by someone suffering from a similar type finger deformity.

Significant advantages to the invention are its low cost, providing the patient with a comfortable support for a painful condition; and, for patient's with toe deformities, allowing the patient to wear ordinary shoes and socks and engage in a wide range of activities in which they might not otherwise be able to participate.

In accordance with the invention, a support 10 is provided which is worn on the patient's foot to correct the deformity. The toe brace or support comprises a band 16 of material which over or under the deformed toe as circumstances warrant. At opposite ends of the band are loops of material formed into respective sleeves 12, 14. These sleeves are fitted onto toes on each side of the deformed toe. The band of material suppresses the distortion of the toe so when the support is in place, the deformed toe is held in place in a more normal side-by-side toe position rather than overlying an adjacent toe. The material is pliable material, but also has sufficient rigidity that when the support is worn, the toe or finger cannot assume its deformed position, but rather is restrained in its proper configuration with respect to the other toes or fingers.

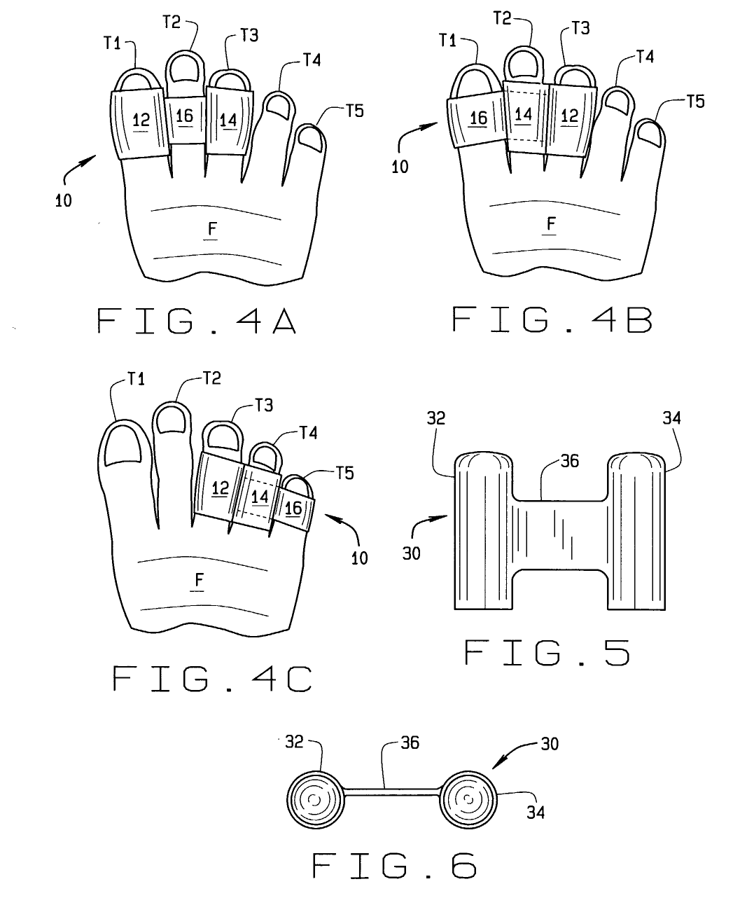

In FIG. 4A, toe T2 is the deformed toe. Support 10 is worn by sliding sleeve 12 onto big toe T1 of the patient's foot, and sleeve 14 onto middle toe T3 of the foot. As shown in FIG. 4A, the support is worn with band 16 fitting over the deformed toe. The material comprising the band suppresses movement of toe T2 to return to its position overlying an adjacent toe (toe T1 in FIG. 1A). Rather, toe T2 is now constrained to extend generally side-by-side with toe T1 and the other toes of the foot.

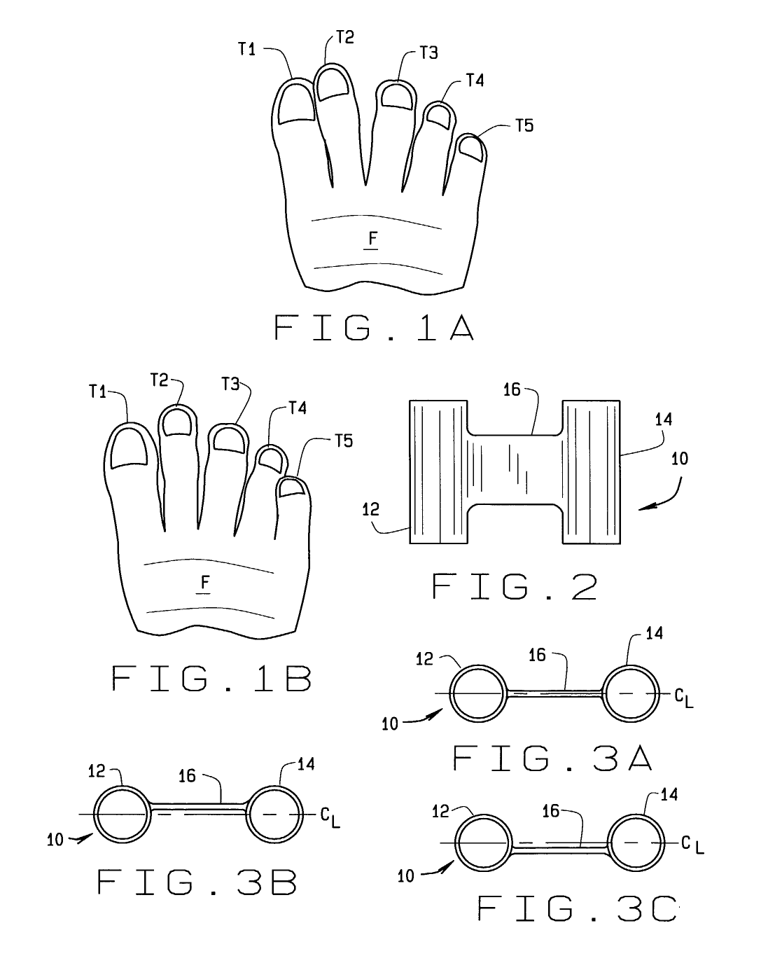

Referring to FIGS. 3A-3C, the type of support 10 used to restrain the deformed toe is a function of the degree of deformation of the toe. For example, if toe T2 is only slightly deformed, the support shown in FIG. 3A may be used. Here, the web of material forming band 16 extends between the sleeves 12, 14, above the centerline CL of the sleeves. If the toe is somewhat more deformed, the support 10 shown in FIG. 3B is used. Here, the web of material forming band 16 extends between sleeves 12, 14 at the location of their centerline. Or, for a more severe deformity, the support 10 of FIG. 3C is used. Now, the band 16 extends between the sleeves below their centerline. It will be noted that these supports are described for use with an overlying toe. Depending upon which support is used, progressively more force is exerted on the deformed toe to keep it in a side-by-side position extending generally parallel with the other toes. Regardless of which support is used, the length of band 16 is sufficient to bridge the width of the deformed toe so the toe is not pinched or otherwise stressed. Those skilled in the art will appreciate that these supports can also be used for an underlying toe condition, but in reverse order depending upon the severity of the condition.

Besides the position of band 16 relative of material forming the sleeves, the force exerted on the deformed toe to straighten it can be controlled by the type of material from which band 16 is made. Preferably, band 16, and the sleeves, are of a pliable material. Depending upon the pliability of the material, more or less force is exerted on the toe. The less pliable the material, the more force is placed upon the toe to maintain it straight. In addition to its pliability, the material is also preferably wrinkle free. This prevents the toes from being irritated, when the support is worn, and particularly prevents sores. In this regard, and as shown in FIG. 10, the inside of the sleeves 12 and 14 can have a lining comprised of a thin layer 18 of a padding material. If the skin is sensitive to the particular material from which support 10 is made, the padding is useful is allowing the patient to wear the support. Similarly, a layer 20 of padding is incorporated on the underside of band 16 to prevent irritation. The material from the support is made is washable so the support can be cleaned after wearing and reused. Alternately, the support is disposed of after being wearing and the support is provided to the treating physician or patient in a container holding a number of supports so there is a ready supply for use by the patient.

Support 10 is useful to correct deformation of any toe. As previously described with respect to FIG. 4A, the support is readily worn to correct deformation of any of the middle toes T2-T4 of the foot. The support can, however, also be used if the deformed toe is the big or little toe T1 or T5. As shown in FIG. 4B, to correct deformation of the big toe, sleeve 14 of support 10 is worn on adjacent toe T2. Band 16 is then wrapped over, around, and under the big toe. Sleeve 12 is then placed over the middle toe T3. Similarly, if the deformed toe is the little toe, sleeve 14 is worn on adjacent toe T4. Band 16 is now wrapped over, around, and under little toe T5. Sleeve 12 is again worn on middle toe T3. In each instance, the width of band 16 is sufficient to comfortably extend over the deformed toe and between the end sleeves so sufficient force is exerted on the deformed toe to position it without pinching or constricting the other toes.

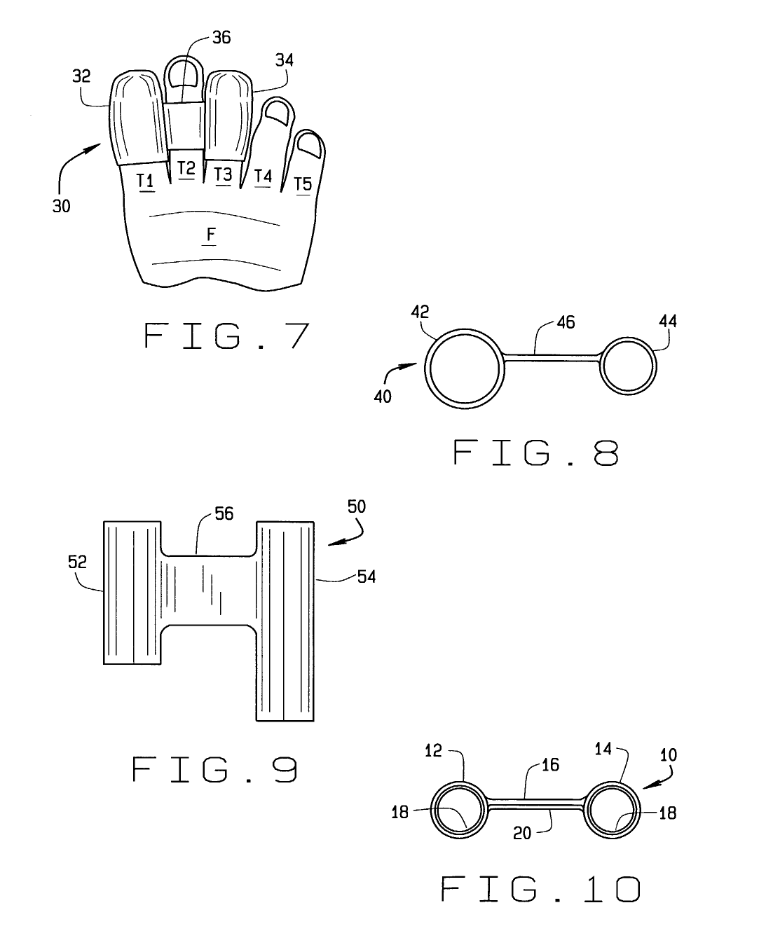

Referring to FIGS. 5-7, another embodiment of the support is indicated generally 30. This support includes end sleeves 32, 34, and a band 36 of material extending between the sleeves. The support is constructed in the same manner as support 10, except now, the outer end of the respective sleeves are closed or capped. The outer end of each of the patient's toes T1 and T3 is now covered by the capped end of the sleeves as shown in FIG. 7. The capped ends of the sleeves can also be padded for added comfort. It will be understood by those skilled in the art that support 30 can be made in a manner similar to the constructions shown in FIGS. 3A-3C, and FIG. 10.

Another construction of the support is indicated generally 40 in FIG. 8. Here, one of the sleeves (sleeve 42) is larger in diameter than the other sleeve (sleeve 44). A band 46 of material extends between the sleeves. Support 40 can be worn, for example, where the patient has an exceptionally large toe so that if support 10 were used, it would not comfortably over the person's big toe.

Finally, another embodiment of the support is indicated generally 50 in FIG. 9. Here, one of the sleeves (sleeve 54) is longer than the other sleeve (52). A band 56 of material extends between the sleeves. This version of the support is for use by patient's with exceptionally long toes, for example.

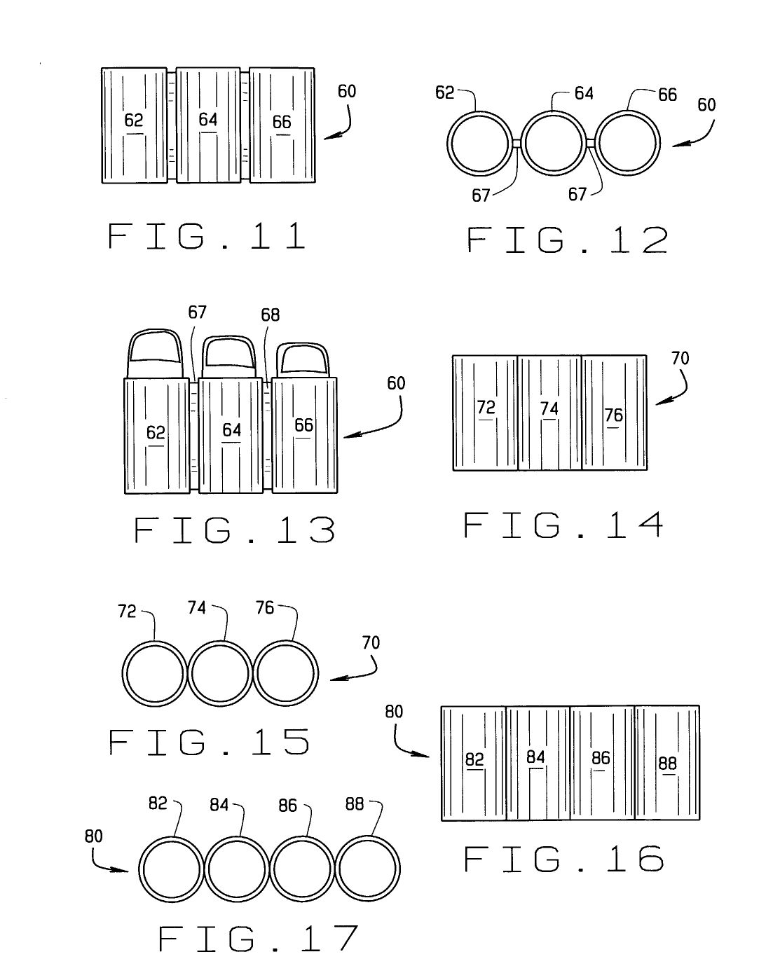

While not shown, those skilled in the art will understand that other toe supports within the scope of this invention are possible. For example, the width of the band between the sleeves can span the width of two or more toes. Such a support could be used where a toe has been amputated, or two adjacent toes have deformities. In addition, as shown in FIGS. 11 and 12, a support 60 can include more than two sleeves. Here, the support includes sleeves 62, 64, and 66. Sleeve 64 fits over the deformed toe, and sleeves 62, 66 fit over the adjacent toes on each side of the deformed toe. This is as shown in FIG. 13. The adjacent sleeves are attached together using respective bands 67, 68. Depending upon the degree of deformity, and the consequent amount of force required to restrain the deformed toe, sleeve 64 can be of a different (i.e., stiffer) material than the other two sleeves, and the connecting bands 67, 68 can be formed as shown in FIGS. 3A-3C.

For some conditions, where the deformity may be particularly severe, the restraining force produced by the bands between the sleeves may not be sufficient to restrain the toe or finger. Now, and as shown in FIGS. 14 and 15, a support 70 has sleeves 72, 74, and 76 which are connected directly together. There are no bands connecting together adjacent sleeves. A similar construction is shown in FIGS. 16 and 17 for a support 80 in which four bands 82, 84, 86, and 88 are connected directly to each other.

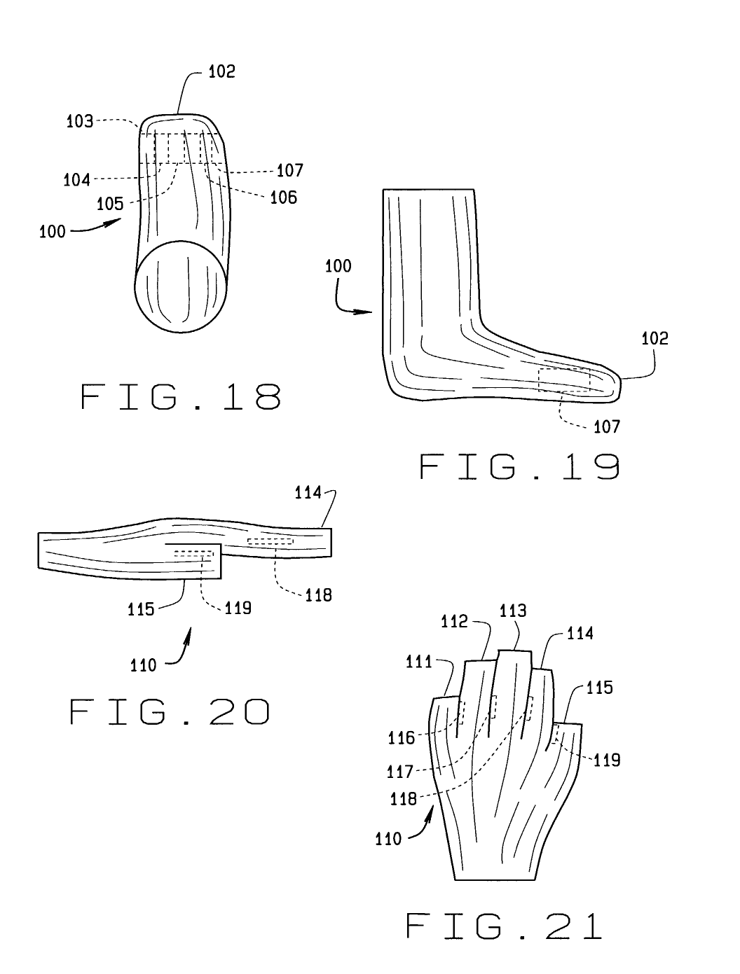

Referring to FIGS. 18 and 19, and FIGS. 21 and 22, the strap of the present invention can be incorporated into a sock 100 or a glove 110 to be worn by the patient with the toe or finger deformity. While shown as a full sock in the drawings, sock 100 can be an anklet as well. Regardless, the sleeves, or sleeves and bands previously described can be woven into the toe end 102 of sock 100. In FIG. 18, five sleeves 103-107, one for each toe are formed in the toe of the sock. There could be as few as two sleeves and the attachment band sewn inside the sock in accordance with the previously described embodiments, up to the five sleeves shown in FIG. 18.

In FIGS. 20 and 21, glove 110 is shown to include a sleeve 111-115 for each finger of the hand; although it will be understood by those skilled in the art that a glove having only sleeves for three of the fingers could also be provided. The sleeves could be attached to each other along their entire length. Or, bands such as the bands 116-119 shown in the drawings could also be provided. The material from which the glove is fabricated, and the strength of the bans, if they are provided, produces sufficient restraining force to hold the deformed finger in the desired position.

In view of the foregoing, it will be seen that the several objects of the invention are achieved and other advantageous results are obtained.

As various changes could be made in the above constructions without departing from the scope of the invention, it is intended that all matter contained in the above description or shown in the accompanying drawings shall be interpreted as illustrative and not in a limiting sense.

1. A support worn by a person on their hand or foot to correct a deformity to a digit (finger or toe) of the hand or foot comprising:
a first sleeve of a support material sized to fit over a digit to one side of the deformed digit;
a second sleeve of the material sized to fit over another digit to one side of the deformed digit; and,
a band of material extending between the two sleeves, the band of material fitting about the deformed digit when the support is worn to hold the deformed digit in a preferred position in which it extends generally side-by-side with the other digits.

2. The support of claim 1 wherein the length of the band of material extending between the sleeves is sufficient to bridge at least the width of the deformed digit.

3. The support of claim 2 in which the length of the band of material extending between the sleeves is sufficient to bridge the width of more than one digit.

4. The support of claim 1 in which the band of material comprises a third sleeve fitting over the deformed digit.

5. The support of claim 4 further including a first band of material connecting the first sleeve to the third sleeve, and a second band of material connecting the second sleeve to the third sleeve.

6. The support of claim 1 in which the first and second sleeves are of the same diameter.

7. The support of claim 6 in which one of the sleeves is larger in diameter than the other.

8. The support of claim 1 in which the first and second sleeves are of the same length.

9. The support of claim 8 in which one of the sleeves is longer than the other.

10. The support of claim 1 in which an outer end of the sleeves, when the support is worn, is enclosed so to form a cap over the ends of the respective digits on which the sleeves are fitted.

11. The support of claim 1 in which the inside of each sleeve is lined with a padding material to prevent irritation of the person's skin when wearing the support.

12. The support of claim 11 further including a padding on the underside of the band to also prevent irritation of the person's skin.

13. The support of claim 1 which is incorporated into the toe of a sock worn by the person.

14. The support of claim 1 which is incorporated into a glove worn by the person.

15. A support worn by a person on their hand or foot to correct a deformity to a digit (finger or toe) of the hand or foot comprising a first sleeve of a support material sized to fit over a digit to one side of the deformed digit, a second sleeve of the material sized to fit over another digit on the opposite one side of the deformed digit, and a third sleeve of the material positioned between the first and second sleeves, the third sleeve fitting over the deformed digit when the support is worn to hold the deformed digit in a position in which it does not overlay the adjacent digit but extends generally side-by-side with the other digits.

16. The support of claim 15 further including a band of material extending between the first sleeve and the third sleeve and the second sleeve and the third sleeve.

17. The support of claim 14 further including a fourth sleeve in which a digit is received.

18. A support sock worn by a person having a deformed toe comprising:
a first sleeve of a support material sized to fit over a toe to one side of the deformed toe, a second sleeve of the material sized to fit over a toe on the opposite one of the deformed toe, and a third sleeve of the material positioned between the first and second sleeves, the third sleeve fitting over the deformed toe when the support is worn to hold the deformed toe in a position in which it does not overlay the adjacent toe but extends generally side-by-side with the other toes, the sleeves being sewn into a toe of the sock for the person to insert their toes into the sleeves when putting on the sock.

19. The sock of claim 18 further including at least one additional sleeve sewn into the toe of the sock for the person to insert another of their toes into the additional sleeve when the sock is put on.

20. A glove worm by a person having a deformed finger comprising.
a first sleeve of a support material sized to fit over a finger to one side of the deformed finger, a second sleeve of the material sized to fit over a finger on the opposite one side of the deformed finger, and a third sleeve of the material positioned between the first and second sleeves, the third sleeve fitting over the deformed finger when the support is worn to hold the deformed finger in a position in which it does not overlay the adjacent finger but extends generally side-by-side with the other fingers, the sleeves forming a glove worn on the person's hand with the person inserting their fingers into the sleeves when putting on the glove.

21. The glove of claim 20 further including at least one additional sleeve into which the person inserts another of their fingers when the glove is put on.

22. The glove of claim 20 further including respective bands of material extending between adjacent sleeves of the glove.

23. A method of treating a deformed toe on a person's foot comprising:
fitting a support onto the person's foot, the support including a pair of sleeves sized to fit over two of the person's toes which are not deformed, a band of material extending between the sleeves;
placing the band over the deformed toe for the band to maintain the deformed toe in a generally side-by-side position relative to the other toes; and,
having the person wear the support for an extended period of time sufficient for the deformity to be corrected.

24. The method of claim 23 in which the band of material comprises a third sleeve into which the deformed toe is inserted.

25. A method of treating a deformed finger on a person's hand comprising:
fitting a support onto the person's hand, the support including a pair of sleeves sized to fit over two of the person's fingers which are not deformed, a band of material extending between the sleeves;
placing the band over the deformed finger for the band to maintain the deformed finger in a generally side-by-side position relative to the other fingers; and,
having the person wear the support for an extended period of time sufficient for the deformity to be corrected.

26. The method of claim 25 in which the band of material comprises a third sleeve into which the deformed finger is inserted.
